(19) United States
(12) Patent Application Publication
(10) Pub. No.: US 2018/0250287 A1
(43) Pub. Date: Sep. 6, 2018

(54) ABUSE DETERRENT OPIOID FORMULATIONS

(71) Applicant: Tactus Therapeutics, Inc., Manhattan Beach, CA (US)

(72) Inventors: A. Mike Schwarz, Manhattan Beach, CA (US); Daniel R. Kimbell, Glendale, CA (US); Charles William Stark, Rancho Palos Verdes, CA (US)

(21) Appl. No.: 15/913,792

(22) Filed: Mar. 6, 2018

Related U.S. Application Data
(60) Provisional application No. 62/467,776, filed on Mar. 6, 2017.

(51) Int. Cl. A61K 31/485; A61K 31/4468; A61K 9/06; A61K 9/00; A61K 47/32; A61P 25/04

(52) U.S. Cl. CPC A61K 31/485; A61K 31/4468; A61P 25/04; A61K 9/0031; A61K 47/32; A61K 9/06

(57) ABSTRACT

An abuse deterrent opioid formulation for rectal use. The formulation contains a therapeutically effective amount of the opioid buprenorphine or salts and homologs thereof; and either a gel with a diminishing agent or a suppository base with a diminishing agent into which the opioid buprenorphine is mixed.

### CROSS REFERENCE TO RELATED APPLICATIONS

This application claims priority to prior filed provisional patent application No. 62/467,776, filed on Mar. 6, 2017, entitled “ABUSE DETERRENT OPIOID FORMULATIONS.”

### FIELD OF THE INVENTION

The invention relates to opioid analgesics, and more particular to abuse deterrent analgesic opioid formulations.

### BACKGROUND OF THE INVENTION

Opioid analgesics are some of the oldest and most widely used forms of pain relieving drugs known to man. Opium is the oldest form and is a gummy extract from the opium poppy. Opium was widely used in ancient civilizations including the Sumerians, Assyrians, Egyptians, and others and its use in pain relief was recognized well before then. Opioid analgesics continue to play a central role in pain management including for moderate-to-severe pain, whether chronic or acute.

Opioids are often classified into three classes: natural derivatives occurring in opium such as morphine and codeine; partially synthetic derivatives, including dihydrocodeine, hydrocodone, (aka dihydrocodeinone), thebacon (aka dihydrocodeinone enol acetate), heroin (diacetylmorphine), hydromorphone, oxycodone, oxymorphone, and buprenorphine; and synthetic compounds such as methadone, levorphanol, butorphanol, fentanyl, sufentanil, pethidine (aka meperidine and Demerol), and tapentadol. This listing is quite incomplete as there are literally hundreds of analgesic opioids. A more comprehensive but still not fully encyclopedic listing is presented further below.

In general, opioids may be administered for therapy by any suitable route. Depending on the opioid, suitable routes of administration may include oral, rectal, nasal, inhalation of aerosols or particulates, topical (administered or occurring elsewhere in the body than the mouth and alimentary canal) including buccal (mouth or cheek), sublingual (under the tongue), transdermal (across the skin), vaginal, rectal, intravesical (into the bladder) and parenteral [including subcutaneous, intramuscular, intravenous, intrasternal (injection in the bone marrow of the sternum), intrathecal (in the space under the arachnoid membrane of the brain or spinal cord), epidural (on or around the dura mater, in particular introduced into the space around the dura mater of the spinal cord) and intradermal (below the skin)].

While not the most popular route of administration, it is known that the rectal administration of certain opioids such as morphine via suppositories can provide effective pain relief, e.g., in infants who might otherwise not swallow a pill, or for those who have difficulty swallowing or with poor veins where intravenous injections is difficult or undesirable. Indeed, there are some people who cannot take opioids via the oral or IV routes. For example, some cancer patients have great difficulty swallowing pills. Other, whose veins are damaged or collapsed, are not ideal candidates for the IV route of administration of opioids. When morphine is provided in suppositories, this is typically done by placing a morphine pill in the middle of a mass of a thick and oily substance, such as solidified cocoa butter, hard fat, gelatinous mixture, and macrogols (polyethylene glycol (PEG)), etc. As the cocoa butter or other material naturally heats up by its placement in the rectum, the morphine pill will dissolve into the carrier substance, which carrier substance laced with morphine will then be absorbed through veins in the patient's rectum. However, morphine (and other opioid) suppositories are quite easy to abuse because user can easily remove the opioid pill from the suppository and then abuse it by other routes (e.g. orally, by IV, by smoking or “chasing”, etc.) and/or concentrate the opioids so that a high dose of the opioid can then be taken.

Also, opioids, even if used properly, can have some unwanted side effects. Opioid induced constipation and nausea are major problems for many who consume opioids. Opioid-induced constipation (OIC) is the most common gastrointestinal adverse effect of opioid use and cause a significant reduction in the quality of life. Other common gastrointestinal effects of opioids are nausea, vomiting, abdominal pain, bloating, and cramping. The prevalence of OIC increases with increased duration of use of opioid analgesics. Treatment satisfaction with opioids decreases when OIC develops and many patients tend to discontinue opioid therapy when they develop constipation. Chronic pain is described as persistent pain for more than 3 months. The prevalence of chronic widespread pain in the general population was about 10%-15%. In patients with chronic noncancer pain, the prevalence of OIC varies from 41% to 81%. The most common indication for opioid use in noncancer pain is musculoskeletal pain, including back pain, degenerative joint disease, fibromyalgia and headache. In the United States, 4% of adults are taking chronic opioid therapy, chiefly for noncancer pain. The Centers for Disease Control and Prevention (CDC) estimates that over prescription of opioids by healthcare providers is the reason for opioid-related overdosing (http://www.cdc.gov/drugoverdose/dataloverdose.html). Almost 90% of patients with moderate to severe pain are treated with opioids.

Opioids have pharmacological effects throughout the gastrointestinal tract. They decrease gastric emptying and stimulate pyloric tone, resulting in anorexia, nausea and vomiting. Inhibition of propulsion and increased fluid absorption in the small and large intestine result in delayed absorption of medications, hard dry stools, constipation, straining, sense of incomplete rectal evacuation, bloating and abdominal distention. Other motor effects are increased anal sphincter tone and pyloric tone, impaired reflex relaxation in response to rectal distention, and increased amplitude of nonpropulsive segmental contractions. These effects result in impaired ability to evacuate the bowel, as well as abdominal spasm, cramps and pain. Decreased gastric, biliary, pancreatic and intestinal secretions interfere with digestion.

The clinical presentation of OIC does not differ from that of functional constipation except that the constipation occurs with opioid treatment. Prospective studies have generally identified OIC on the basis of the Rome III criteria definition of constipation. The most common symptoms used as inclusion criteria in these trials are less than three bowel movements (BMs)/week, straining, hard stools and sensation of incomplete evacuation. OIC can occur even at low dosages of opioids and can occur at any time after initiation of opioid therapy. Nausea, vomiting and gastroesophageal reflux are the other symptoms associated with OIC.

Thus, it is clear that standard routes of opioid administration cause major opioid induced constipation, as well as opioid induced nausea. One advantage of the rectal route of administration of the opioid formulation of the invention is that it circumvents the stomach and upper intestine and may result in a reduction in the opioid induced constipation and opioid induced nausea.

The opioid fentanyl is administered via transdermal patches for chronic pain management, and can be administered by intravenous injection, by an intranasal spray sublingually, and as fentanyl lozenges which are a solid formulation of fentanyl citrate on a stick in the form of a lollipop that dissolves slowly in the mouth for transmucosal absorption. There are also buccal tables or patches.

Regardless of how opioids are administered to the patient's body, opioids will ultimately enter the patient's bloodstream where they will have their desired analgesics affects. Unfortunately, irrespective of the route of administration, the longer a person uses an opioid, the greater the risk that the user may become addicted to that opioid, with some commonly used opioids having greater potential for addiction than other opioids, including for example codeine, fentanyl, hydrocodone (multiple brands), hydromorphone (Dilaudid®), levorphanol (Levo-Dromoran®), meperidine (Demerol®), methadone (Dolophine®), morphine sulfate, oxycodone, (Roxicodone, Xtampza ER™) propoxyphene (Darvon®) tapentadol (Nucynta®); Non-Narcotic analgesics such as Fioricet®, Fiorinal®, Soma compound®, tramadol (Ultram®), Agonist-Antagonists, buprenorphene (Buprenex®), Belbuca™, Probuphine®, butorphanol (Stadol®), dezocine (Dalgan®), nalbuphine (Nubain®), pentazocine (Talwin®); Antagonists such as nalmefene (Revex®), naltrexone, naloxone (Narcan®), and others, such as Suboxone® (sublingual/buccal film containing buprenorphine and naloxone) which is used for the treatment of opioid dependence, and ziconotide (Prialt®). The branded time released version of oxycodone by Purdue Pharma is Oxycontin® has been found to have serious risk of addiction.

In recent years, the abuse of prescription opioids and illegal opioids has grown dramatically, creating grave health, societal, economic, and law enforcement problems. Indeed, in the not too distant past and even today, opioids such as oxycodone and hydrocodone were rather liberally prescribed and became readily available on the black market. In some areas, so called “pill mills” (a term used primarily by law enforcement to describe a doctor, clinic or pharmacy that is prescribing or dispensing powerful narcotics inappropriately or for non-medical reasons) have become such a danger that physicians and pharmacists have even started to be prosecuted for murder. The statistics are dire, and according the Centers for Disease Control and Prevention, in 2010, about 12 million Americans (age 12 or older) reported nonmedical use of prescription painkillers in the past year; nearly half a million emergency department visits in 2009 were due to people misusing or abusing prescription painkillers; nonmedical use of prescription painkillers costs health insurers up to $72.5 billion annually in direct health care costs; and enough prescription painkillers were prescribed in 2010 to medicate every American adult around-the-clock for a month. The U.S. opioid epidemic endures, and drug overdose deaths nearly tripled during 1999-2014. According to the Division of Unintentional Injury Prevention, National Center for Injury Prevention and Control, CDC, among 47,055 drug overdose deaths that occurred in 2014 in the United States, 28,647 (60.9%) involved an opioid. The trend worsened in 2015, where drug overdoses accounted for 52,404 U.S. deaths, including 33,091 (63.1%) that involved an opioid. The drug overdose death rate increased significantly from 12.3 per 100,000 population in 2010 to 16.3 per 100,000 in 2015.

Fentanyl, a Schedule II drug, is a powerful synthetic opioid pain medication with a rapid onset and short duration of action, and is estimated to be between 50 and 100 times as potent as morphine. Fentanyl is more effective than morphine because it is more easily dissolved in fats or lipids. Because of this, Fentanyl is fast-acting and easily penetrates to the central nervous system. Fentanyl is prescribed for breakthrough pain, such as cancer pain, and is available in fentanyl transdermal patches, fentanyl lollipop to be absorbed buccally, dissolving tablets, and a sublingual spray which are absorbed through the cell lining inside the mouth. In palliative care, transdermal fentanyl has a role for patients already stabilized on other opioids who have persistent swallowing problems and cannot tolerate other parenteral routes such as subcutaneous administration, people with moderate to severe renal failure, and those who have troublesome side effects of oral morphine, hydromorphone, or oxycodone.

Unfortunately, as with many other opioids, illicit recreational use of pharmaceutical fentanyl and its more than a dozen different analogues is widespread, and there are analogues of fentanyl. When used illicitly, fentanyl is also consumed orally, smoked, chased, snorted, or injected. Fentanyl is sometimes even sold as heroin, often leading to overdoses. Fentanyl is sometimes sold on the black market in the form of transdermal fentanyl patches such as Duragesic®, diverted from legitimate medical supplies. Addicts have been known to disassemble the transdermal patches as aspirate out (e.g., with a syringe) the fentanyl from several patches, and then inject the thusly retrieved fentanyl so they can experience a quicker and more powerful fix. This use has led to thousands of overdose deaths each year from 2000 to 2015. The biological effects of the fentanyl analogues are similar to those of heroin. Fentanyl analogues may be hundreds of times more potent than street heroin, and tend to produce significantly more respiratory depression, making it much more dangerous than heroin to users. Illegal use of fentanyl by those without opiate tolerance can be very dangerous and has resulted in numerous deaths, and even those with opiate tolerances are at high risk for overdoses. Indeed, some heroin dealers mix fentanyl powder with heroin to increase potency or compensate for low-quality heroin. Fentanyl also finds its way into fake oxycodone pills and even cocaine. Once the fentanyl is in the user's system, it is extremely difficult to stop its course because of the nature of absorption. Because of the extremely high strength of pure fentanyl powder (measured in microgram doses), it is very difficult to dilute appropriately, and often the resulting mixture may be far too strong and, therefore, very dangerous.

Buprenorphine, a Schedule III drug, was developed as a safer alternative to methadone for treating addiction to heroin and painkillers. Two formulations of the drug are used in such therapy. Subutex® and Buprenex®, which are pure buprenorphine, are designed to be used in the initial stages of addiction treatment. Buprenex® is a liquid form intended solely for intravenous or intramuscular injection. This form of the drug is not approved for the treatment of opioid addiction, but only for treatment of acute pain. Buprenex® has been used in the U.S. for relief of acute pain since the 1980s. Relative to morphine, it is a fairly good analgesic. Suboxone®, offered by Invidior Inc., which contains buprenorphine and naloxone as an anti-abuse component, is designed to be used in the maintenance stage of treatment. Both drugs are available in a variety of dosing formulations, namely sublingual films and tablets, injection solutions, intradermal implants, and transdermal extended release patches and both are intended to block the effects of opiates while reducing opiate cravings and easing withdrawal symptoms. Buprenorphine is the only opiate addiction therapy drug that can be prescribed in a physician's office; others must be dispensed in a clinic. When Suboxone in its pill form is taken correctly by being dissolved under the tongue, the naloxone is not absorbed and is inactive. But if a Suboxone pill is inappropriately crushed and injected, the naloxone will be active and will put the addict into immediate withdrawal. If the naloxone was not in the drug, the buprenorphine alone could still be abused, to produce a high, or euphoria. In other words, naloxone is a safety feature, to discourage injection of the medication. This method of distribution via a doctor's office is advantageous to many opiate addiction therapy patients because it is more convenient and less stigmatizing than clinic-based therapy, which typically involves methadone. Unfortunately, pure forms of buprenorphine such as Subutex in its currently available liquid for injection formulation are increasingly being abused, and despite safety measures in place to guard against diversion of the drug, illegal distribution and abuse of buprenorphine have been reported.

In response to the opioid crisis, besides more tightly monitoring and controlling the distribution of legal medical opioids to pain centers, physicians, and pharmacies, some pharmaceutical companies have introduced opioids with abuse deterrent technologies to make such opioids more resistant to abuse. For example, some addicts will attempt to crush time released pills to more quickly release the opioid (e.g., oxycodone) and manufacturers have thus introduced reformulated pills to make them more difficult to crush, and if crushed, more difficult to either ingest, smoke, chase, snort, or inject. In addition, unpleasant flavorants are sometime added to render crushed pills less palatable. Also, some products have colorants (e.g. brightly colored permanent staining agents) so that if the drug is abused, e.g., by snorting, the abuser will be left with brightly coloring around his or her nostrils.

Opioid addictions are some of the most difficult addictions to overcome, and opioid addicts go to great lengths to get the opioid drugs they need to satisfy their physical and psychological cravings. Whatever the attempt to discourage opioid abuse, desperate people such as opioid addicts become very creative and actually share information via networks, blog sites, and other routes to circumvent efforts by manufacturers to thwart abuse.

Opioid products can be abused in a variety of ways. For example, they can be swallowed whole, crushed and swallowed, crushed and snorted, crushed and smoked or chased, crushed, dissolved and injected. Smoking heroin or other opioids can involve filling part of a cigarette with powder opioid, applying flame to the end of the cigarette, and smoking it like a normal cigarette. While it is a simple process to smoke opioids, this method of abuse can be quite inefficient because the intense heat of the burning tobacco and the direct flame degrades and destroys most of the opioid, with only a small amount of the opioid being is vaporized and inhaled, wherein the drug can enters the bloodstream. Chasing heroin or other opioids involves placing the opioid in the stem of an opium pipe, or in the middle of a square piece of aluminum foil or spoon, adding heat from the bottom, and inhaling the vapors through a tube. Chasing can result in more of the drug entering the bloodstream. There are many other ways people abuse opioids, including making mixed drinks with alcohol and opioids, which can be extremely dangerous.

As a result, doctors, pharmacists, and manufacturers and distributors of opioids have started to make more efforts to limit the amount of opioids that can be prescribed to individuals. Consequently, the availability of black market prescription opioids has shrunk in recent years, and the prices have risen significantly, in a typical supply and demand relationship. This has resulted in some unintended and dire consequences, including but not limited to the following. First, the black market has been flooded with fake prescription opioids, many of which do not contain the noted opioid or noted dose. This can result in terrible outcomes, including serious illness and death by overdose. Second, the demand for and abuse of heroin have increased dramatically. This is because heroin is in many cases much more available and far less expensive than black market prescription opioids, such as oxycodone. Using heroin is quite dangerous because the quality and quantity of heroin (not to mention other possible drugs) in a given supply is never certain, and death and illness related to heroin overdose is a serious problem. Third, people who actually have a need for strong opioid analgesics are finding it more difficult to obtain prescription opioids from their physicians because ethical physicians are becoming more hesitant and cautious before prescribing opioid analgesics, fearing that they may be responsible if a patient suffers from abuse or an overdose.

Because opioid products are often manipulated for purposes of abuse by different routes of administration or to defeat extended-release (ER) properties, most abuse-deterrent technologies developed so far are intended to make manipulation more difficult or to make abuse of the manipulated product less attractive or less rewarding. Unfortunately, these technologies have not yet proven successful at deterring the most common form of abuse-swallowing a number of intact capsules or tablets to achieve a feeling of euphoria. The fact that a product has abuse-deterrent properties does not equate to elimination of abuse. Rather, it means that the risk of abuse is lower than it would be without such properties. Because opioid products must in the end be able to deliver the opioid to the patient in some relatively convenient manner, there may always be some abuse of these products.

The science of abuse deterrence is relatively new, and both the formulation technologies and the analytical, clinical, and statistical methods for evaluating those technologies are rapidly evolving. Based on the evolving nature of the field, a flexible, adaptive approach to the evaluation and labeling of potentially abuse-deterrent products needs to be taken, and methods for evaluating the abuse-deterrent properties of opioids may have to be adapted based on the characteristics of those products and the anticipated routes of abuse. The development of an abuse-deterrent opioid product should be guided by the need to reduce the abuse known or expected to occur with similar products.

The federal Food and Drug Administration considers it critical to address the problem of opioid abuse while seeking to ensure that patients in pain have appropriate access to opioid products. Moreover, the FDA has a policy that opioids without abuse-deterrent properties remain available for use in some clinical settings, for example, patients in hospice care and with difficulty swallowing may need access to opioid products that are in solution or that can be crushed. As used herein the term “abuse-deterrent” means formulations with properties that to meaningfully deter abuse, even if they do not fully prevent abuse. The term “abuse” is defined as the intentional, non-therapeutic use of a drug product or substance, even once, to achieve a desirable psychological or physiological effect without intent to mitigate pain and suffering related to a medical condition or following surgical intervention that is known or is expected to cause moderate to severe pain.

. Misuse is not the same as abuse. Misuse refers to the intentional therapeutic use of a drug product in an inappropriate way and specifically excludes the definition of abuse.

As noted above, opioid products can be abused in a number of ways. For example, they can be swallowed whole, crushed and swallowed, crushed and snorted, crushed and smoked, or crushed, dissolved and injected. Abuse-deterrent technologies should target known or expected routes of abuse relevant to the proposed product. According to the FDA Guidance document “Abuse-Deterrent Opioids—Evaluation and Labeling Guidance for Industry”, from April 2015, as a general framework, abuse-deterrent formulations can currently be categorized as follows:

1. Physical/chemical barriers. Physical barriers can prevent chewing, crushing, cutting, grating, or grinding of the dosage form. Chemical barriers, such as gelling agents, can resist extraction of the opioid using common solvents like water, simulated biological media (e.g., to mimic conditions in the gut), alcohol, or other organic solvents. Physical and chemical barriers can limit drug release following mechanical manipulation, or change the physical form of a drug, rendering it less amenable to abuse.

2. Agonist/antagonist combinations. An opioid antagonist can be added to interfere with, reduce, or defeat the euphoria associated with abuse. The antagonist can be sequestered and released only upon manipulation of the product. For example, a drug product can be formulated such that the substance that acts as an antagonist is not clinically active when the product is swallowed, but becomes active if the product is crushed and injected or snorted.

3. Aversion. Substances can be added to the product to produce an unpleasant effect if the dosage form is manipulated or is used at a higher dosage than directed. For example, the formulation can include a substance irritating to the nasal mucosa if ground and snorted.

4. Delivery System (including use of depot injectable formulations and implants.) Certain drug release designs or the method of drug delivery can offer resistance to abuse. For example, sustained-release depot injectable formulation or a subcutaneous implant may be difficult to manipulate, as they can be administered in the clinic to monitor compliance and would be difficult to transfer/sell.

For the reasons noted above, responsible physicians, pharmacists, and other associated health care providers would like to be able to prescribe and fill opioid prescriptions with new opioid formulations that are less subject to abuse and addiction, and there accordingly remains a need for same.

### SUMMARY OF THE INVENTION

The present invention is an abuse deterrent opioid formulation for rectal use, comprising: a therapeutically effective amount of an opioid and pharmaceutically acceptable salts, derivatives, homologs or analogs thereof; a carrier; and a formulation diminishing agent, which diminishing agent renders the opioid formulation difficult to inject through an intravenous needle and if heated, changes the consistency of the abuse deterrent opioid formulation, further rendering the abuse deterrent opioid formulation more difficult to misuse effectively administer either orally or by injection.

In another embodiment the invention provides an abuse deterrent opioid formulation for rectal use, comprising: a therapeutically effective amount of the opioid buprenorphine or salts and homologs thereof; a gel carrier comprises a gelling agent and a solvent; and a heat-activated formulation diminishing agent comprising thermoplastic resin, wherein opioid formulation is provided in a predetermined dose in a syringe, and the opioid formulation, if heated above a boiling point of the solvent in the gel, will become thicker and will not effectively allow the opioid contained in the opioid formulation to be isolated from the gel.

In yet another embodiment the invention provides an abuse deterrent opioid formulation for rectal use, comprising: a therapeutically effective amount of the opioid buprenorphine or salts and homologs thereof; and a gel carrier comprises a gelling agent and a solvent, wherein the opioid formulation, if heated above a boiling point of the solvent in the gel, will become thicker and will not cause the opioid contained in the opioid formulation to separate out from the gel.

In still a further embodiment the invention provides an abuse deterrent opioid formulation for rectal use, comprising: a therapeutically effective amount of the opioid buprenorphine or salts and homologs thereof; a gel carrier comprises a gelling agent and a solvent; and a heat-activated formulation diminishing agent comprising thermoplastic resin, wherein opioid formulation is provided in a predetermined dose in a syringe, and the opioid formulation, if heated above a boiling point of the solvent in the gel, will become thicker and will not cause the opioid contained in the opioid formulation to separate out from the gel.

In a yet further embodiment the invention provides an abuse deterrent opioid formulation for rectal use, comprising: a therapeutically effective amount of the opioid buprenorphine or salts and homologs thereof; and a carrier comprises a suppository base, wherein the opioid formulation, even if heated above a melting point of the suppository base, will not cause the buprenorphine contained in the opioid formulation to separate out from the suppository base. Other abuse deterrent means can be incorporated including the addition of destructive agents which will help deter the opioid from being swallowed, snorted, smoke, or injected.

These and other features of the invention are described below.

### BRIEF DESCRIPTION OF THE DRAWINGS

FIG. 1 is a photograph of one exemplary gel formulation comprising Carbomer 940 and water as mixed up in a beaker.

FIG. 2 is a photograph of the gel of FIG. 1 placed on a spoon and partially heated up with a torch to mimic what would be a drug abuser's attempt to liquefy and/or chase the gel formulation to obtain any opioid to be contained therein.

FIG. 3 is a photograph of the gel of FIG. 2 when further heated, showing the gel formulation as dehydrated, shrunken, and blackened.

FIG. 4 is a photograph of another exemplary gel formulation comprising Carbomer 940 and water plus a diminishing agent in the form of finely chopped HDPE plastic film and placed on a spoon to mimic a drug abuser's attempt to liquefy and/or chase the gel formulation to obtain any opioid to be contained therein.

FIG. 5 is a photograph of the gel with the HDPE plastic diminishing agent of FIG. 4 after being heated up with a torch to mimic a drug abuser's attempt to liquefy and/or chase the gel formulation to obtain any opioid to be contained therein, showing the gel formulation dehydrated, shrunken, and blackened.

FIG. 6 is a photograph of another exemplary gel formulation comprising Carbomer 940 and water plus a diminishing agent in the form of finely ground expanded polystyrene foam.

FIG. 7 is a photograph of the gel of FIG. 6 placed on a spoon and partially heated up with a torch to mimic a drug abuser's attempt to liquefy and/or chase the gel formulation to obtain any opioid to be contained therein.

FIG. 8 is a photograph of the gel with the finely ground expanded polystyrene foam plastic diminishing agent of FIG. 7 after being further heated, showing the gel formulation as dehydrated, shrunken, and blackened.

FIG. 9 is a view of a standard #2 disposable cone coffee filter.

FIG. 10 shows the disposable cone coffee filter of FIG. 9 placed in a filter basket and used to try to extract the opioid from a gel of the invention which has been diluted with a solvent.

FIG. 11, there is shown a view of an exemplary blunt tip syringe that can be used to deliver an opioid containing gel formulation of the invention

FIG. 12 is a photograph of an exemplary suppository of the invention containing buprenorphine in a suppository base.

### DETAILED DESCRIPTION

In one embodiment, the invention provides an abuse deterrent opioid formulation for rectal use, comprising a therapeutically effective amount of an opioid (and pharmaceutically acceptable salts, derivatives, homologs or analogs thereof); a carrier that is preferably a gel but can be a liquid, a foam, a lotion, a cream, an ointment, or a paste; and formulation diminishing agent. All ingredients are mixed together in a uniform mixture. The diminishing agent, upon being heat-activated, renders the opioid formulation more difficult to administer and more difficult from which to extract the opioid.

In the abuse deterrent opioid formulation for rectal use, any number of different opioids can be used, including but not limited to all agents under CII and CIII substances per 21 CFR §§ 1300-1399, such as the following: alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethyl-thiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacyl-morphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phena-doxone, phenomorphan, phenazocine, phenoperidine, piminodine, piritramide, propheptazine, promedol, properidine, propoxyphene, sufentanil, tilidine, tramadol, mixtures of any of the foregoing, pharmaceutically acceptable salts, derivatives, homologs or analogs of any of the foregoing, and the like.

The phrase “pharmaceutically acceptable salts, derivatives, homologs or analogs” is intended to convey any pharmaceutically acceptable tautomer, salt, pro-drug, hydrate, solvate, metabolite or other compound which, upon administration to the subject, is capable of providing (directly or indirectly) the compound concerned or a physiologically (e.g., analgesically) active compound, metabolite or residue thereof. An example of a suitable derivative is an ester formed from reaction of an OH or SH group with a suitable carboxylic acid, for example C₁₋₃ alkyl-CO₂H, and HO₂C (CH₂—)ₙ—CO₂H (where n is 1-10 such as 1, 2, 3, 4, 5, 6, 7, 8, 9, 10, but preferably 1-4), and CO₂H—CH₂-phenyl. Thus, the active compounds may be in crystalline form, either as the free compounds or as solvates (e.g. hydrates). Methods of solvation are generally known within the art.

The salts of the active compounds of the invention are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid.

The term “metabolite” includes any compound into which the active agents can be converted in vivo once administered to the subject. Examples of such metabolites are glucuronides, sulphates and hydroxylates. Metabolites may be active, partially active, more active or inactive.

It will be understood that active agents as described herein may exist in tautomeric forms. The term “tautomer” is used herein in its broadest sense to include compounds capable of existing in a state of equilibrium between two isomeric forms. Such compounds may differ in the bond connecting two atoms or groups and the position of these atoms or groups in the compound. A specific example is keto-enol tautomerism.

The compounds of the present invention may be electrically neutral or may take the form of polycations, having associated anions for electrical neutrality. Suitable associated anions include sulfate, tartrate, citrate, chloride, nitrate, nitrite, phosphate, perchlorate, halosulfonate or trihalomethylsulfonate.

The two preferred opioids in the abuse deterrent opioid formulations for rectal use of the invention comprise fentanyl and salts thereof, and buprenorphine and salts thereof. In the case of fentanyl, it is not taken orally as it largely broken down by the patient's stomach acids, and is poorly available to the patient's bloodstream. Accordingly, it is difficult to abuse by oral administration. The features of the invention that make the abuse determine fentanyl formulation of the invention difficult to use, such as by injection and smoking, helps make this formulation less likely to be abused.

The feature of providing the opioid in rectal formulations may help reduce the potential for addiction as many patients will find this route of administration less convenient and more unpleasant than other routes, such as oral pills. The idea of a slower rate of absorption, yet a sustained coverage and protection from “breakthrough pain” is a therapeutic advantage for the patient population for the formulation of the invention. Such patients are not looking for the quick rush high, but would like to be continually pain free without the debilitating side effects of being “too doped up”. Moreover, for those, e.g., cancer patients, who have difficulty swallowing pills, or whose veins are damaged or collapsed and are thus not ideal candidates for the IV route of administration of opioids, the rectal route of administration is desirable. Also, while it is relatively simple for a person to swallow more opioid pills at one time or in a day than are prescribed, it is much more difficult for a user to take multiple large volume rectal doses, and doing so would be expected to be unpleasant for most users. Thus, most users are less likely to take more opioids than prescribed, and people seeking opioids to meet an opioid addiction, but who are not otherwise in great pain, would be expected to be less likely to abuse the rectal opioid formulation of the invention would instead try to secure easy to abuse or rush inducing opioid formulations.

As noted above, the carrier in the abuse deterrent opioid formulation is preferably a gel but can be a foam, a liquid, a lotion, a cream, an ointment, a suppository base, or a less preferably, a paste.

Turning first to pharmaceutical liquids, liquids have a very low viscosity and while they may carry opioids, they are not ideal for carrying opioids for rectal use. Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Next, a lotion is a low-viscosity topical preparation intended for application to unbroken skin. By contrast, creams and gels have higher viscosity. Most lotions are oil-in-water emulsions using a substance such as cetearyl alcohol to keep the emulsion together, but there are also water-in-oil lotions. Creams are semi-solid emulsions of oil and water. They are divided into two types: oil-in-water (O/W) creams which are composed of small droplets of oil dispersed in a continuous phase, and water-in-oil (W/O) creams which are composed of small droplets of water dispersed in a continuous oily phase. Oil-in-water creams are less greasy and more easily washed off using water. On the other hand, while more difficult to handle, many drugs which are incorporated into creams are hydrophobic (including fentanyl which has a high lipophilicity) and will be released more readily from a water-in-oil cream than an oil-in-water cream. Ointments are homogeneous, viscous, semi-solid preparations, most commonly a greasy, thick oil (oil 80%-water 20%) with a high viscosity. The different types of ointment bases include: hydrocarbon bases, e.g., hard paraffin, soft paraffin, microcrystalline wax and ceresine; absorption bases, e.g. wool fat, beeswax; water-soluble bases, e.g., macrogols 200, 300, 400; emulsifying bases, e.g. emulsifying wax, cetrimide; and vegetable oils, e.g., cocoa butter, olive oil, coconut oil, sesame oil, almond oil and peanut oil, or petroleum based oils (in case food allergies are of concern. Pastes in their basic pharmaceutical form consist of a fatty base (e.g., petroleum jelly) and at least 25% solid substance (e.g., zinc oxide). Pastes are the semisolid preparations intended for external application to the skin. They are usually thick and do not melt at normal temperature so are not an ideal choice as a carrier for the rectal opioid formulation of the invention.

In the case of suppositories, a variety of base materials are used. One is the fat- or oil-type base, which must melt at body temperature to release its medication, such as cocoa butter and cocoa butter substitutes. A second is the glycerin-gelatin base suppository, which absorbs water and dissolves to release its medication. A third is the water-soluble or water-miscible polymers and surface-active agents such a polyethylene glycol bases. A fourth is a group of bases containing disintegrating agents, natural gums, effervescent agents, collagen, fibrin, hydrogels, etc. Regardless of the type of suppository base used, they should all preferably possess the following qualities: A. Chemically and physically stable under normal conditions of use and storage, B. Nonreactive and compatible with a wide variety of drugs and auxiliary agents; C. Free from objectionable odor(s), which in the case of the current invention will be modified); D. An aesthetically appealing appearance; E. Nontoxic, nonsensitizing, and nonirritating to sensitive tissues; F. Expansion—contraction characteristics such that it shrinks just enough on cooling so that it releases easily from suppository molds; G. Melts or dissolves in the intended body orifice to release the drug; H. Nonbinding of drugs; I. Mixes with or absorbs some water; J. Viscosity low enough when melted to pour easily but high enough to suspend particles of solid drug; and K. Some wetting and/or emulsifying properties so that it will spread, disperse in, and release the active ingredient(s) at the administration site.

In one exemplary embodiment of the invention, the base used is PCCA #30-1056 (Fatty Acid Base, which is hydrogenated vegetable oil and PEG-8 Disterate, offered by Professional Compounding Centers of America (PCCA), of Houston, Tex.

Turning lastly to gels as carriers for the abuse deterrent opioid formulation for rectal use, such gels comprise a solvent (including but not limited to water, an alcohol, and/or propylene glycol), and at least one gelling agent. The gelling agents include but not limited to at least one of acacia, alginic acid, bentonite, a carbomer, carboxymethyl cellulose, ethylcellulose, gelatin, hydroxyethyl cellulose, hydroxypropyl cellulose, magnesium aluminum silicate, methylcellulose, poloxamers, polyvinyl alcohol, sodium alginate, tragacanth, and xanthan gum. Goods results are achieved with carbomers as carbomers have an excellent ability to cross-link with the solvent even at low concentrations, and even in lower weight percentages, carbomers can form thick gels. For rectal use, the gel of the invention will ideally have a viscosity in the range of 10,000 to 500,000 centipoise, and more preferably in the range of 25,000 to 250,000 centipoise, and even more preferably in the range of 40,000 to 80,000 centipose. Gels with such viscosity ranges are readily injectable into patient's rectum via a syringe with wide tips and will aid in the opioid formulation be retained in the rectum where the opioid may be absorbed over time by vasculature in the patient's rectum.

The feature of incorporating the opioid in the carrier by itself makes it difficult for users to successfully extract the opioid from the carrier. For example, where the carrier is a gel, the gelling agent will be cross-linked with the solvent- e.g., water and/or oil. Thus, heating the gel formulation (for example above a boiling point of the solvent in the gel) in an attempt to liquefy the gel and drive opioid laden solvent from the gel will not succeed because instead of driving opioid laden liquid from the gel (e.g., where it can be inhaled), the solvent in the gel will evaporate off, leaving a stiffer gel. Such a stiffer opioid containing gel will remain difficult to smoke, chase, inject, or eat. The formulation diminishing agent further enhances the already good abuse deterrent aspects of the abuse deterrent analgesic opioid formulations as follows. The formulation diminishing agent preferably comprises at least one polymer, and is more preferably a thermoplastic polymer, including but not limited to polyvinyl chloride (PVC), polyethylene (PE), polystyrene (PS), and polypropylene (PP). Regardless of the exact nature of the formulation diminishing agent, the formulation diminishing agent is preferably provided as small and finely divided granules mixed into the gel. If a user attempts to inject the opioid formulation through a needle, the small and finely divided granules will tend to clog the syringe needle and thereby interfere with injection of the opioid formulation. Other types of formulation diminishing agent can be used to help prevent the formulation from being injected, including appropriately sized silicon dioxide particles (sand grains) and calcium carbonate particles. The formulation diminishing agent will preferably be non-absorbable and is eliminated intact into the feces and is also large enough to clog injection needles.

By way of example, IV needles typically used in needle exchange programs for IV drug users are 28 gauge (with an interior needle diameter of about 0.184 mm and an outer diameter of 0.3620 mm.) If needles get much larger, they excessively damage the user's veins upon injection. By way of example, a size 23 needle has an inner diameter of 0.337 mm and an outer diameter of 0.6414 mm. Thus, the granule size of the formulation diminishing agent will be selected to be above the inner diameter of the needles, preferably larger than about 0.2 millimeter in diameter, for example 0.2 mm to 0.5 mm. Where the formulation diminishing agent is a thermoplastic polymer, and is heated above a threshold temperate, e.g., a melting point or decomposition temperature thermoplastic polymer, the thermoplastic polymer will decompose and vent strong and unpleasant smelling and/or noxious fumes and the thermoplastic polymer will also become hardened, brittle, and/or blackened. As the opioid formulation is heated, these unpleasant smelling and/or noxious fumes are released along with the solvent gassing off from the gel formulation, and thus, to the extent there even a small percentage of the opioid is undamaged and actually gases off, the presence of the unpleasant smelling and/or noxious fumes will tend to dissuade users from trying to inhale the opioid formulation. As the formulation diminishing agent in the gel decomposes and burns and hardens, this will irreversibly damage the opioid formulation and rendering it less able to be used in any manner. If the formulation diminishing agent is heated above a threshold temperature (e.g., a decomposition temperature of the formulation diminishing agent), then the formulation diminishing agent will decompose and release gases and cause the opioid formulation to at least one of scorch and harden, rendering the heated opioid formulation more difficult to at least one of (a) dissolve in more of the original solvent in the opioid formulations or in another solvent, (b) separate out the opioid from the heated opioid formulation, (c) process the heated opioid formulation into an intravenously injectable form, (d) smoke or chase the heated opioid formulation, (e) snort the heated opioid formulation up nostrils, (f) consume the heated opioid formulation buccally, (g) swallow the heated opioid formulation, and (h) absorb the heated opioid formulation through the skin and mucous membrane in any part of the human body.

The thusly damaged opioid formulation will be more difficult to use orally, transmucosally, rectally or vaginally including via suppository, subcutaneously, intravenously, intramuscularly, intraperitoneally, intragastrically, intranasally, intrathecally, transdermally or intestinally. Of course, even when undamaged, the gel cannot be easily abusable intranasally, by IV or by intramuscularly (I.M.)

Unpleasant flavorant and/or odorant which are offensive if smelled or tasted by a user are optionally included in the abuse deterrent opioid formulation. Such additional agents will help deter abuser from attempting to take the abuse deterrent opioid formulation orally or heating same, as the offensive smell will be released. The unpleasant flavorant and/or odorant can comprise, for example, appropriate quantities of at least one of butyric acid (with a strong feces and vomit smell) and indole (which has a feces smell when present in large concentrations.)

The abuse deterrent opioid formulation for rectal use of claim 3, wherein in order to render the opioid formulation fluid enough to inject through a syringe into a person's veins, at least X times more solvent will need to be added to the opioid formulation, wherein X is between 10 and 1000, thereby highly diluting the opioid formulation and making it difficult to abuse by injection.

In a preferred embodiment the abuse deterrent opioid formulation for rectal (or vaginal) use is provided in a predetermined dose in a syringe with a plunger and a rectal injection tip for injection of the opioid formulation into a patient's rectum. The total volume of the abuse deterrent opioid formulation can range in volume from about 5 ml to about 15 ml, and the amount (whether by weight or volume) of the selected opioid in the formulation will be targeted to be in the same general range of use when the opioid is medically used intravenously. Indeed, veins in the rectum provide excellent absorption of medications deposited therein, and when for the example the opioid is mixed into gel carrier, the gel will tend to adhere to the walls of the rectum thereby permitting the opioid to travel into the veins and into the patient's bloodstream. Indeed, certain sections of venous supply to the rectum bypass the liver, and therefore if the abuse deterrent opioid formulation gel is deposited in this region, there is excellent bioavailability of the opioid in the body.

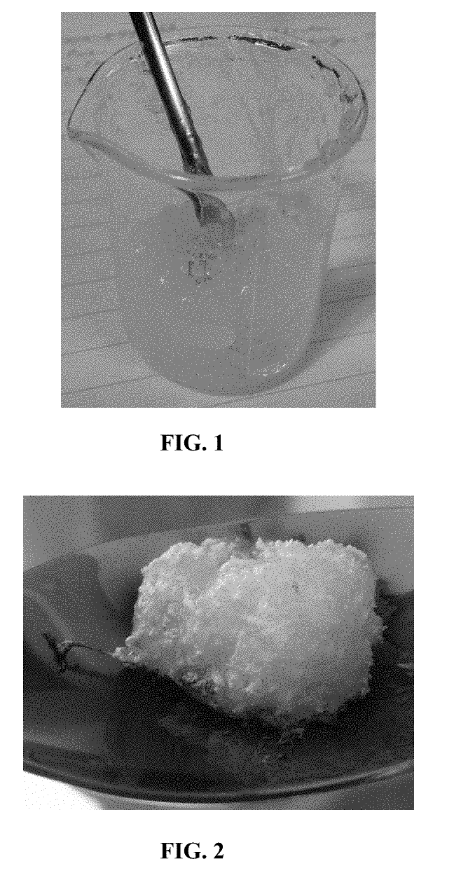

FIG. 1 is a photograph of one exemplary gel formulation comprising the gelling agent carbomer 940 and water as mixed up in a beaker. No opioid is present in this exemplary gel formulation. This exemplary gel formulation comprises about 2.0 g of carbomer 940 with 30 ml of water, which is stirred and allowed to set up so that the water permeates the carbomer 940 to form a stiff gel having a viscosity of about 40,000 to 80,000 centipose.

FIG. 2 is a photograph of a dollop of the gel of FIG. 1 placed on a spoon. The spoon was heated up at its bottom surface with a torch to mimic what would be a drug abuser's attempt to liquefy the gel to extract any opioid to be contained therein, and/or chase the gel formulation so that it can be smoked. As can be seen, because of the intense heat from the torch, the bottom of the gel has started to harden and blacken, but no solvent (water) is driven from the gel. During the heating process, strong and unpleasant smelling but non-carcinogenic fumes from the decomposing plastic carbomer 940 are given off.

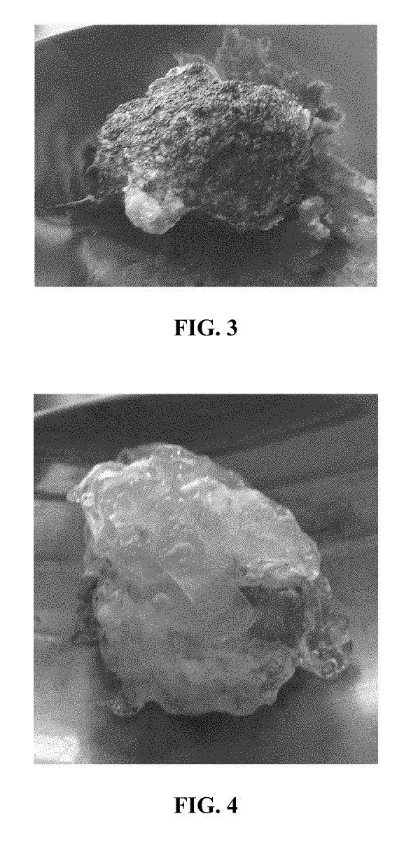

FIG. 3 is a photograph of the dollop of gel of FIG. 2 when further heated, showing the gel formulation as dehydrated, shrunken, and with the water (solvent) driven away and with the carbomer 940 blackened. This heated exemplary gel formulation resists efforts to extract any solvent and solvent carrying the opioid, and thus would resist efforts to extract and/or concentrate the opioid present in the gel where such opioid bearing solvent could be taken orally, injected into the veins, taken sublingually, or otherwise administered other than as intended. Moreover, because of the decomposition of the plastic carbomer 940 that occurs upon heating, the gel of the invention would not be conducive to being smoked or chased.

FIG. 4 is a photograph of another exemplary gel formulation comprising Carbomer 940 and water plus a diminishing agent in the form of finely chopped HDPE plastic film and placed on a spoon to mimic a drug abuser's attempt to liquefy and/or chase the gel formulation to obtain any opioid to be contained therein. No opioid is present in this exemplary gel formulation. This exemplary gel formulation comprises about 2.0 g of carbomer 940 with 30 ml of water, which is stirred and allowed to set up so that the water permeates the carbomer 940 to form a stiff gel having a viscosity of about 40,000 to 80,000 centipose. The amount of finely chopped HDPE plastic film in the formulation can range in weight percentage of the gel by about 0.1% to about 10%.

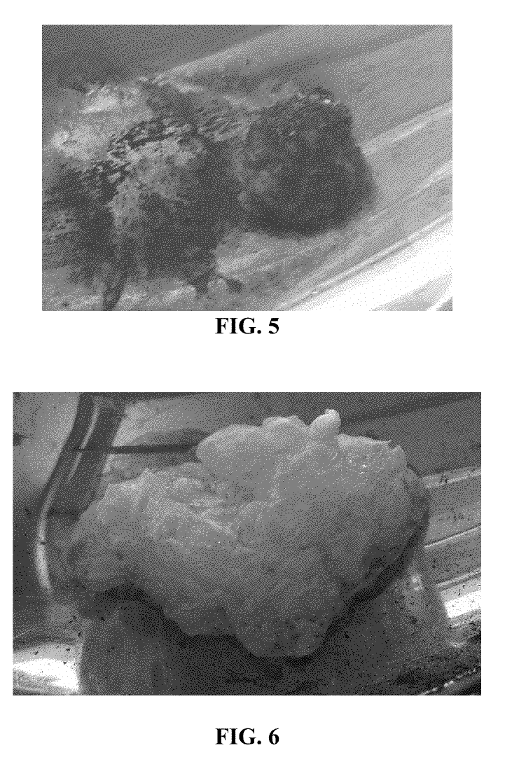

FIG. 5 is a photograph of the gel with the HDPE plastic diminishing agent of FIG. 4 after being heated up with a torch to mimic a drug abuser's attempt to liquefy and/or chase the gel formulation to obtain any opioid to be contained therein, showing the gel formulation as dehydrated, shrunken, and with the water (solvent) driven away and with the carbomer 940 and the HDPE plastic blackened. This heated exemplary gel formulation resists efforts to extract any solvent and solvent carrying the opioid, and thus would resist efforts to extract and/or concentrate the opioid present in the gel where such opioid bearing solvent could be taken orally, injected into the veins, taken sublingually, or otherwise administered other than as intended. Moreover, because of the decomposition of the plastic carbomer 940 that occurs upon heating, the gel of the invention would not be conducive to being smoked or chased.

FIG. 6 is a photograph of another exemplary gel formulation comprising Carbomer 940 and water plus a diminishing agent in the form of finely ground expanded polystyrene plastic (Styrofoam) and placed on a spoon. No opioid is present in this exemplary gel formulation. This exemplary gel formulation comprises about 2.0 g of carbomer 940 with 30 ml of water, which is stirred and allowed to set up so that the water permeates the carbomer 940 to form a stiff gel having a viscosity of about 40,000 to 80,000 centipoise. The amount of finely ground expanded polystyrene plastic in the formulation can range in weight percentage of the gel by about 0.1% to about 10%. This image shows the gel formulation after being slightly heated on a spoon.

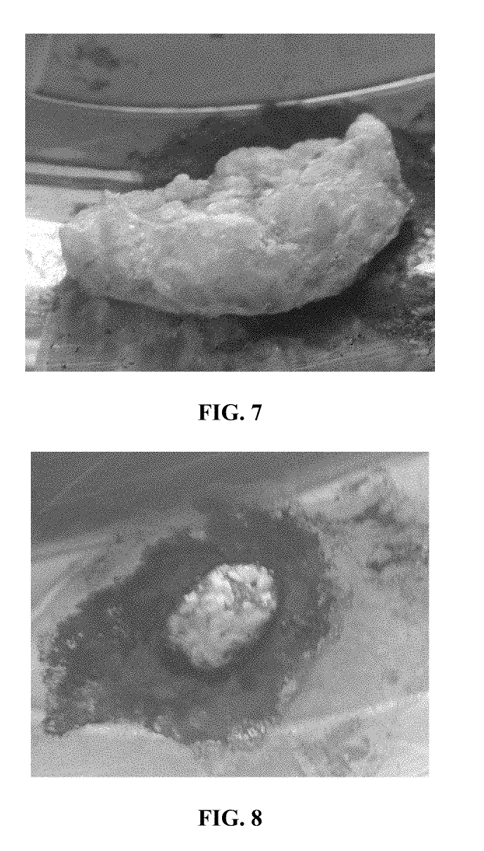

FIG. 7 is a photograph of the gel of FIG. 6 placed on a spoon and heated up further with a torch to mimic a drug abuser's attempt to liquefy and/or chase the gel formulation to obtain any opioid to be contained therein. This picture showing the gel formulation as slightly more dehydrated, shrunken, and with the water (solvent) driven away and with the carbomer 940 and the expanded polystyrene blackened at its bottom where it contact the spoon.

FIG. 8 is a photograph of the gel with the finely ground expanded polystyrene foam plastic diminishing agent of FIG. 7 after being further heated, showing the gel formulation completely dehydrated, shrunken, and blackened. As with the other exemplary embodiments of the formulation of the invention, this heated exemplary gel formulation resists efforts to extract any solvent and solvent carrying the opioid, and thus would resist efforts to extract and/or concentrate the opioid present in the gel where such opioid bearing solvent could be taken orally, injected into the veins, taken sublingually, or otherwise administered other than as intended. Moreover, because of the decomposition of the plastic carbomer 940 plus the additional expanded polystyrene that occurs upon heating, the gel of the invention would not be conducive to being smoked or chased.

As noted above, the use of heat in an attempt to access to the opioid in the gel formulations of the invention simply does not work. Heating does not result in the opioid be driven out of the solvent in the gel, and the gases that emanate are laced with not only vapors from the solvent that has boiled off, but also strong and unpleasant fumes of the decomposing plastic of the gelling agent.

To further enhance the deterrence of the opioid formulation, an unpleasant flavorant(s), odorant(s), and/or bitterant(s) (or bittering agent) are preferentially added to the formulation. Such unpleasant flavorant(s) and/or odorant(s) can comprise, for example, appropriate quantities of at least one of butyric acid and indole. Products laced with indole (with the smell of feces), and butyric acid (with a distinct and bad smell of vomitus or feces) are not something that most users would be expected to wish to smell, much less put in their mouths. When heated, the flavorants/odorants will release even more of the unpleasant flavors and smells, further acting as a deterrent to abuse of the opioid formulations of the invention. Some common bitterants include denatonium, usually available as denatonium benzoate, sucrose octaacetate, and quercetin, to name a few commonly used bitterants. While safe, these bitterants make swallowing of products containing them extremely difficult, thus further deterring oral consumption of the opioid.

As noted above, opioid abusers sometimes attempt to use common solvents like water, simulated biological media (e.g., to mimic conditions in the gut), alcohol, or other organic solvents in an attempt to abuse certain opioid formulations. For example, they may attempt to mix a solvent into crushed opioids pills so that the resulting solution can be injected or swallowed, the idea being to get quick acting effects and bypass the intended administration route.

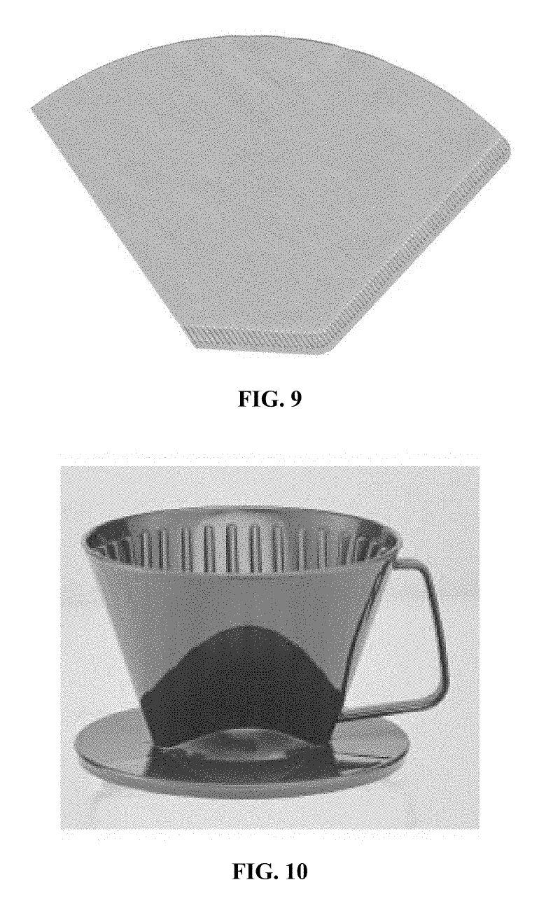

In an experiment, a gel of the invention (containing 2 g of carbomer 940 as gelling agent and 30 ml of water) was further diluted with 100 ml of water in an attempt to separate the solvent from the gelling agent (to “ungel” the formulation) and run the diluted gel through a standard #2 disposable cone coffee filter (FIG. 9) placed in a filter basket (FIG. 10). Coffee filters of paper are made from about 100 g/m2 filter paper. The raw materials (pulp) for the filter paper are coarse long fiber, often from fast growing trees. Typically disposable coffee filters are made up of filaments approximately 20 micrometers wide, which allow particles through that are less than approximately 10 to 15 micrometers. While the addition of 100 ml of water resulted in a less viscous gel, when the now less viscous gel was placed in #2 cone drip coffee filter, gel collected on the inside of coffee filter and the coffee filter become clogged. No water dripped out of the coffee filter through the filter basket. Thus, use of additional water added to the gel of the invention does not appear to function to defeat the gelling agent to prevent dilution and extraction of any substance(s) contained therein, e.g., opioids.

In a further experiment, 50 ml of vodka (40% ethanol) was added to a gel of the invention (containing 2 g of carbomer 940 as gelling agent and 30 ml of water). With vigorous stirring, the vodka incorporated into the gel with a less viscous gel resulting. While the addition of 50 ml of vodka resulted in a less viscous gel, when the now less viscous gel was placed in #2 cone drip coffee filter, gel collected on the inside of coffee filter and the coffee filter become clogged. No vodka/water dripped out of the coffee filter through the filter basket. Thus, use of vodka added to the gel of the invention does not appear to function to defeat the gelling agent to prevent dilution and extraction of any substance(s) contained therein, e.g., opioids.

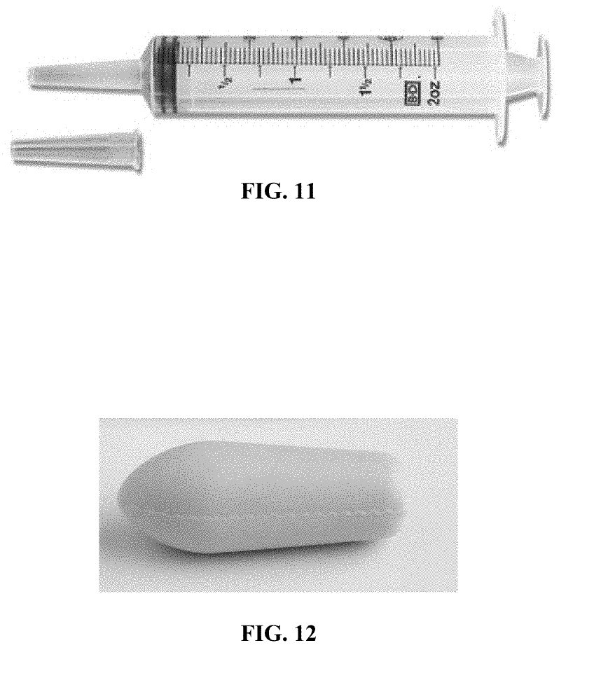

Turning to FIG. 11, there is shown a view of an exemplary blunt tip syringe that can be used to deliver an opioid containing gel formulation of the invention. The volume of the opioid containing gel formulation can vary as desired, but may vary between about 5 to 50 ml, and more preferably between about 10 ml to about 20 ml. For example, it is desirable that syringes can come with predetermined volumes of gel and opioid, e.g., 100, 250, 500, 1000, and 2000 micrograms of fentanyl in 10 ml of gel, with the dose selected based on the patient's body medical need, body weight, age, sex, etc. The tip of the syringe can be lubricated before insertion into a patient rectum, or be delivered with a pre-lubricated tip. Once the tip of the syringe is inserted into the patient's rectum, the plunger of the syringe will be depressed to force the opioid containing gel formulation of the invention into the rectum. The opioid containing gel formulation will thus be deposited in the rectum and the syringe tip will be withdrawn. The now nearly empty syringe will be discarded in a safe and approved manner. The opioid contain gel formulation will tend to deposit on the inner walls of the rectum where the opioid in the formulation will travel to and be absorbed into the vasculature of rectum and will enter the bloodstream over time, where the therapeutic effects of the opioid will be experienced. Depending on how the gel is formulated, the rate at which the opioid will travel through the gel and enter the bloodstream can be carefully designed and controlled to provide either quick and short acting relief (e.g., for breakthrough pain), or a sustained release formulation for pain relief that last over a long period of time.

Turning to FIG. 12, there is shown a photograph of an exemplary suppository of the invention containing buprenorphine in a suppository base. The exemplary suppository can be made with a variety of suppository base materials, and in this exemplary embodiment is made using the base PCCA #30-1056 (Fatty Acid Base, which is hydrogenated vegetable oil and PEG-8 Disterate), offered by Professional Compounding Centers of America (PCCA), of Houston, Tex.

A variety of buprenorphine strengths can be provided, such as suppositories containing 1 mg, 2 mg, 4 mg. and 8 mg per suppository, depending on the patient's particular needs. The size of suppository can also vary depending on the size of patient, with suppositories being sized at about 1 g, 2 g or 4 g, with the size of the suppository depends on the size of the recipient—1 g for infants, 2 g for children and 4 g for adults being a general rule.

To further prevent abuse, a diminishing agent can be included in the suppositories. Examples of diminishing agents include HDPE (in the form of tiny beads or finely chopped HDPE plastic film), and finely ground or tiny pellets of expanded polystyrene plastic (Styrofoam®), or other plastics that burn and blacken when heated. In lieu of polymers as a diminishing agent, appropriately sized ground up tree nuts shells, such as walnut shells, can function both as needle clogging agents and burning agents, which if heated above a smoke point will turn black and foul the formulation. For example, an added benefit of ground tree nut shells is that they have environmental benefits since if they get into waterways, are less deleterious to fish and other aquatic creatures.

A drug abuser can easily swallow many, many opioid pills at once to try to achieve a quick high. However, it would be more difficult for an opioid abuser to dispense many, many syringes full of the opioid formulation of the invention into the rectum. Even if this happens, because of the time release qualities, the result may be time release dosing, rather than the quick hit that many addicts and recreational opioid user desire. Another practical factor that would tend to prevent abuse is the bulky nature of syringes full of the opioid gel formulation of the invention. Such syringes full of the opioid gel formulation will bulky. Unlike the ability to carry 100 pills in a small bottle, 100 syringes take up a lot of room and cannot easily be carried on a person, e.g., in their pocket. Lastly, administering the opioid via the opioid gel formulation of the invention is not particularly pleasant, takes time, and must generally be done while the patient is laying down in the prone position, all which should prevent patients from seeking out this particular formulation for abuse. These considerations also apply to suppositories. They are much larger than pills and thus are not so easy to carry large quantities. Plus, insertion of the rectal suppositories of the invention would likewise occur while the patient is lying down in the prone position or bending over.

To summarize, the opioid containing gel formulations and rectal suppositories of the invention will provide relief to patients who truly require treatment with opioids. Those patients who have difficulty swallowing and thus cannot swallow opioid pills, and those whose veins are damage and have difficulty receiving opioids intravenously, will receive benefits from the rectal (or vaginal) routes of delivery. On the other hand, because of the added inconvenience and antipathy of many patients to the rectal route of administration, those who desire to misuse or abuse opioids are expected to be at least partially deterred from doing. As described above, the abuse deterrent features of the gel formulations and rectal suppositories of the invention will make it more difficult to misuse or abuse compared with other opioid formulations. Thus, health care professional who might be unsure whether a patient is truly in need of opioid therapy for a medical need, or is a drug seeking addict, can more confidently prescribe the opioid formulation of the invention with less concern that the opioid will be abused or misused.

The preferred embodiments of this invention have been disclosed, however, so that one of ordinary skill in the art would recognize that certain modifications would come within the scope of this invention.

What is claimed is:

1. An abuse deterrent opioid formulation for rectal use, comprising:
a therapeutically effective amount of an opioid and pharmaceutically acceptable salts, derivatives, homologs or analogs thereof;
a carrier; and
a formulation diminishing agent, which diminishing agent renders the opioid formulation difficult to inject through a small intravenous needle and if heated, changes the consistency of the abuse deterrent opioid formulation, further rendering the abuse deterrent opioid formulation more difficult to misuse.

2. The abuse deterrent opioid formulation for rectal use of claim 1, wherein the carrier comprises a gel.

3. The abuse deterrent opioid formulation for rectal use of claim 2, wherein the gel comprises a solvent and at least one gelling agent.

4. The abuse deterrent opioid formulation for rectal use of claim 3, wherein the at least one gelling agent is selected from acacia, alginic acid, bentonite, a carbomer, carboxymethyl cellulose, ethylcellulose, gelatin, hydroxyethyl cellulose, hydroxypropyl cellulose, magnesium aluminum silicate, methylcellulose, poloxamers, polyvinyl alcohol, sodium alginate, tragacanth, and xanthan gum.

5. The abuse deterrent opioid formulation for rectal use of claim 3, wherein the solvent is selected from the group consisting of water, an alcohol, and propylene glycol.

6. The abuse deterrent opioid formulation for rectal use of claim 2, wherein the gel has a viscosity in the range of 10,000 to 500,000 centipoise, and more preferably in the range of 25,000 to 250,000 centipoise, and even more preferably in the range of 40,000 to 80,000 centipose.

7. The abuse deterrent opioid formulation for rectal use of claim 1, wherein the formulation diminishing agent comprises polymers particles.

8. The abuse deterrent opioid formulation for rectal use of claim 7, wherein the at least one polymer is a thermoplastic.

9. The abuse deterrent opioid formulation for rectal use of claim 8, wherein the at least one thermoplastic is selected from the group consisting of polyvinyl chloride (PVC), polyethylene (PE), polystyrene (PS), and polypropylene (PP) plastics.

10. The abuse deterrent opioid formulation for rectal use of claim 7, wherein the polymer particles range in size between about 0.2 mm to 0.5 mm.

11. The abuse deterrent opioid formulation for rectal use of claim 1, wherein the opioid is at least one opioid selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethyl-thiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacyl-morphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phena-doxone, phenomorphan, phenazocine, phenoperidine, piminodine, piritramide, propheptazine, promedol, properidine, propoxyphene, sufentanil, tilidine, tramadol, mixtures of any of the foregoing, and pharmaceutically acceptable salts, derivatives, homologs or analogs thereof.

12. The abuse deterrent opioid formulation for rectal use of claim 1, wherein the opioid comprises fentanyl and pharmaceutically acceptable salts, derivatives, homologs or analogs thereof.

13. The abuse deterrent opioid formulation for rectal use of claim 1, wherein the opioid comprises buprenorphine and pharmaceutically acceptable salts, derivatives, homologs or analogs thereof.

14. The abuse deterrent opioid formulation for rectal use of claim 1, further comprising an unpleasant flavorant and/or odorant which are offensive if smelled or tasted by a user.

15. The abuse deterrent opioid formulation for rectal use of claim 1, wherein the unpleasant flavorant and/or odorant comprises at least one of butyric acid and indole.

16. The abuse deterrent opioid formulation for rectal use of claim 2, wherein if the opioid formulation is heated above a boiling point of the solvent in the gel, the gel will become thicker and the opioid in the opioid formulation will not separate out from the gel.

17. The abuse deterrent opioid formulation for rectal use of claim 2, wherein if the formulation diminishing agent is heated above a decomposition temperature of the formulation diminishing agent, the formulation diminishing agent will decompose and release gases and cause the opioid formulation to at least one of scorch and harden, rendering the heated opioid formulation more difficult to at least one of (a) dissolve in more of the original solvent in the opioid formulations or in another solvent, (b) separate out the opioid from the heated opioid formulation, (c) process the heated opioid formulation into an intravenously injectable form, (d) smoke or chase the heated opioid formulation, (e) snort the heated opioid formulation up nostrils, (f) consume the heated opioid formulation buccally, (g) swallow the heated opioid formulation, and (h) absorb the heated opioid formulation through the skin and mucous membrane in any part of the human body.

18. The abuse deterrent opioid formulation for rectal use of claim 3, wherein in order to render the opioid formulation fluid enough to inject through a syringe into a person's veins, at least X times more solvent will need to be added to the opioid formulation, wherein X is between 10 and 1000, thereby highly diluting the opioid formulation and making it difficult to abuse by injection.

19. The abuse deterrent opioid formulation for rectal use of claim 1, wherein the opioid formulation is provided in a predetermined dose in a syringe.

20. The abuse deterrent opioid formulation for rectal use of claim 19, wherein the syringe has plunger and a rectal injection tip for injection of the opioid formulation into a patient's rectum.

21. The abuse deterrent opioid formulation for rectal use of claim 1, further comprising staining colorant or dye which if contact with a user's skin will cause the user skin to become dyed.

22. An abuse deterrent opioid formulation for rectal use, comprising:
a therapeutically effective amount of the opioid buprenorphine or salts and homologs thereof; and
a suppository base.

23. The abuse deterrent opioid formulation for rectal use of claim 22, wherein the suppository base comprises hydrogenated vegetable oil.

24. The abuse deterrent opioid formulation for rectal use of claim 22, wherein the formulation further comprises particles of a diminishing agent, which diminishing agent renders the opioid formulation difficult to inject through a small intravenous needle and if heated, changes the consistency of the abuse deterrent opioid formulation, further rendering the abuse deterrent opioid formulation more difficult to misuse.

25. The abuse deterrent opioid formulation for rectal use of claim 24, wherein the formulation diminishing agent comprises one of polymers and small particles of ground tree nuts.

26. The abuse deterrent opioid formulation for rectal use of 25, wherein the particles range in size between about 0.2 mm to 0.5 mm.

27. The abuse deterrent opioid formulation for rectal use of claim 22, wherein the formulation further comprises a bitterant selected from the group consisting of denatonium, sucrose octaacetate, and quercetin.

28. The abuse deterrent opioid formulation for rectal use of claim 24, wherein the opioid is at least one opioid selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethyl-thiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacyl-morphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phena-doxone, phenomorphan, phenazocine, phenoperidine, piminodine, piritramide, propheptazine, promedol, properidine, propoxyphene, sufentanil, tilidine, tramadol, mixtures of any of the foregoing, and pharmaceutically acceptable salts, derivatives, homologs or analogs thereof.

29. The abuse deterrent opioid formulation for rectal use of claim 24, wherein the opioid is buprenorphine and pharmaceutically acceptable salts, derivatives, homologs or analogs thereof.
